(19) United States
(12) Patent Application Publication
(10) Pub. No.: US 2014/0107461 A1
(43) Pub. Date: Apr. 17, 2014

(54) Adaptive Medical Testing

(71) Applicant: CLINICALBOX, INC., Waltham, MA (US)

(72) Inventors: Salim Afshar, Cambridge, MA (US); Farbod Hagigi, Watertown, MA (US); Murali Menon, Lexington, MA (US)

(73) Assignee: CLINICALBOX, INC., Waltham, MA (US)

(21) Appl. No.: 13/690,432

(22) Filed: Nov. 30, 2012

Related U.S. Application Data
(60) Provisional application No. 61/714,106, filed on Oct. 15, 2012.
(60) Provisional application No. 61/714,112, filed on Oct. 15, 2012.

(51) Int. Cl. A61B 5/00; A61B 7/00; A61B 5/11

(52) U.S. Cl. CPC A61B 5/7246; A61B 5/11; A61B 5/0059; A61B 7/00
USPC 600/407; 600/300; 600/595; 600/586

(57) ABSTRACT

A computer-based system and method improves the efficiency (quality and/or cost) of healthcare services provided to a patient by dynamically adapting the tests that are performed on the patient. Such adaptation may include one or both of selecting the tests to be performed and modifying the contents and/or methods of the tests to be performed. Tests may be adapted based on a wide variety of data, such as any one or more of sensor data, coordination data, clinical data, contextual data, test outcome data, clinical outcome data, and epoch of care data. In particular, tests may be adapted based on data in a model of an epoch of care of the patient so that the tests are selected and/or modified in ways that are tailored to the patient's epoch of care.

### CROSS-REFERENCE TO RELATED APPLICATIONS

This application claims priority from the following patent applications, all of which are incorporated by reference herein:
U.S. Prov. Pat. App. Ser. No. 61/714,106, filed on Oct. 15, 2012, entitled, “Epoch of Care-Centric Healthcare System” (Attorney Docket Number C0008-1001L);
U.S. Prov. Pat. App. Ser. No. 61/714,112, filed on Oct. 15, 2012, entitled, “Adaptive Medical Testing” (Attorney Docket Number C0008-1002L); and
a concurrently-filed U.S. Patent Application entitled, “Epoch of Care-Centric Healthcare System” (Attorney Docket Number C0008-1001).

### BACKGROUND

Healthcare professionals perform a wide variety of tests on patients to diagnose diseases and assess patient outcomes. Tests have been developed to measure nearly every function of the human body, from heart rate to fine motor skills to balance. Tests also use a wide variety of tools, ranging from human “sensors” (e.g., the physician's eyes, ears, and hands) to simple tools (e.g., stethoscopes, thermometers, and blood pressure monitors) to complex machinery (e.g., magnetic resonance imaging (MRI) scanners and computerized tomography (CT) scanners).

Despite the long history of testing in the practice of medicine, the wide variety of testing methods and technology currently available, and the critical role that testing plays in the healthcare industry (both for ensuring the quality of healthcare services and for increasing the efficiency with which such services are provided), testing techniques still suffer from a variety of limitations. For example, tests often are performed on patients even when those tests are not necessary to diagnose the patients' diseases or to assess the patient's outcomes, and tests that would be helpful for diagnostic or assessment purposes often are not performed. One reason for such failures is that tests typically are chosen and formulated for a particular patient based on relatively generic characteristics of the patient which do not enable more accurate selection and refinement of the tests performed on that patient.

What is needed, therefore, are improved systems for selecting and formulating tests to be performed on patients for the purposes of diagnosing and assessing those patients in the course of the provision of healthcare services to those patients.

### SUMMARY

A computer-based system and method improves the efficiency (quality and/or cost) of healthcare services provided to a patient by dynamically adapting the tests that are performed on the patient. Such adaptation may include one or both of selecting the tests to be performed and modifying the contents and/or methods of the tests to be performed. Tests may be adapted based on a wide variety of data, such as any one or more of sensor data, coordination data, clinical data, contextual data, test outcome data, clinical outcome data, and epoch of care data. In particular, tests may be adapted based on data in a model of an epoch of care of the patient so that the tests are selected and/or modified in ways that are tailored to the patient's epoch of care.

Other features and advantages of various aspects and embodiments of the present invention will become apparent from the following description and from the claims.

### BRIEF DESCRIPTION OF THE DRAWINGS

FIG. 1 is a dataflow diagram of a system for dynamically adapting tests applied to patients as part of the provision of healthcare services to those patients according to one embodiment of the present invention.

FIG. 2 is a dataflow diagram of a system for adapting medical tests according to one embodiment of the present invention.

FIG. 3 is a flowchart of a method performed by the system of FIG. 2 according to one embodiment of the present invention.

### DETAILED DESCRIPTION

A computer-based system and method improves the efficiency (quality and/or cost) of healthcare services provided to a patient by dynamically adapting the tests that are performed on the patient. Such adaptation may include one or both of selecting the tests to be performed and modifying the contents and/or methods of the tests to be performed. Tests may be adapted based on a wide variety of data, such as any one or more of sensor data, coordination data, clinical data, contextual data, test outcome data, clinical outcome data, and epoch of care (EOC) data. In particular, tests may be adapted based on data in a model of an epoch of care of the patient so that the tests are selected and/or modified in ways that are tailored to the patient's epoch of care.

Before describing examples of techniques that embodiments of the present invention may use to adapt tests, certain terms used herein will be defined.

The term “test,” as used herein, refers to a test that includes a combination of a method, content, and a domain. A test's method includes the mechanisms that are used to perform the test, such as:
technology that is used to provide input to the user, such as technology that is used to provide test content to and/or receive test input from patients, and technology that is used to modify ambient characteristics of the patient's environment (e.g., room lighting, temperature), whether or not such technology is controlled by the test;
media through which test content is provided to and/or through which test input is received from patients;
qualities of the test presentation (e.g., font size, font color, audio volume, brightness); and
technology that contributes to ambient features of the environment in which the test is performed, such as lights that provide ambient lighting, thermostats that control ambient temperature, and air conditioning that contributes to ambient sound during the performance of the test.

Examples of technology that may be used to provide test content to patients include, but are not limited to, computer monitors, touch screens, speakers, and Braille readers. Examples of technology that may be used to receive test input from patients include, but are not limited to, microphones, touch screens, styluses, keyboards, mice, trackballs, and accelerometers. As the example of the touch screen illustrates, a single device may both provide test content to a patient and receive test input from the patient. Devices for providing test content and/or receiving test input may be connected to or embedded within computing devices, such as desktop computers, laptop computers, tablet computers, and smartphones. A single computing device, such as a tablet computer, may include any number of devices for providing test content to users and any number of devices for receiving test input from users.

The medium or media through which test content is provided to and/or through which test input is received from patients may include, for example, light, sound, haptic stimulus, kinetic energy, thermal energy, and electrical energy.

A test's content includes specific elements of the test that are provided to the patient by the test's method. Examples of test content include, but are not limited to, questions (e.g., displayed in text, spoken aloud by a natural or synthetic voice, or output in Braille by a Braille reader), shapes or other objects, colors, symbols (e.g., letters), non-speech sounds, and instructions (e.g., instructions to lift a weight, hold a tablet computer flat, blow into a tube, or read written letters aloud).

Test input may include any response provided by a patient in response to a test's content. Such input may, but need not, be measured or measurable directly by a machine. Input that is not measurable directly by a machine may be measured by a human and then recorded for possible subsequent input into a computer. Examples of machine-measurable input include, for example, any input that may be provided to a computer peripheral (e.g., input to a keyboard, mouse, touchpad, touch screen, or trackball), microphone input, pressure sensor input, accelerometer input, velocity sensor input, heart rate monitor input, or blood pressure monitor input. Test input may, for example, be provided via devices worn by the patient and/or by devices implanted into the patient. As these examples make clear, test input is not limited to input that is provided under the intentional control of the patient. For example, the patient's blood pressure, as measured by a blood pressure monitor, is an example of test input even though the patient does not intentionally provide the value of the patient's blood pressure to the blood pressure monitor. The same is true, for example, for velocity input as measured by a treadmill, weight input as measured by a scale, and body temperature input as measured by a thermometer.

A test's domain refers to the clinical area being tested by the test. Examples of domains include neurocognition, motor function, vision, hearing, balance, and physiological domains such as the cardio-respiratory domain.

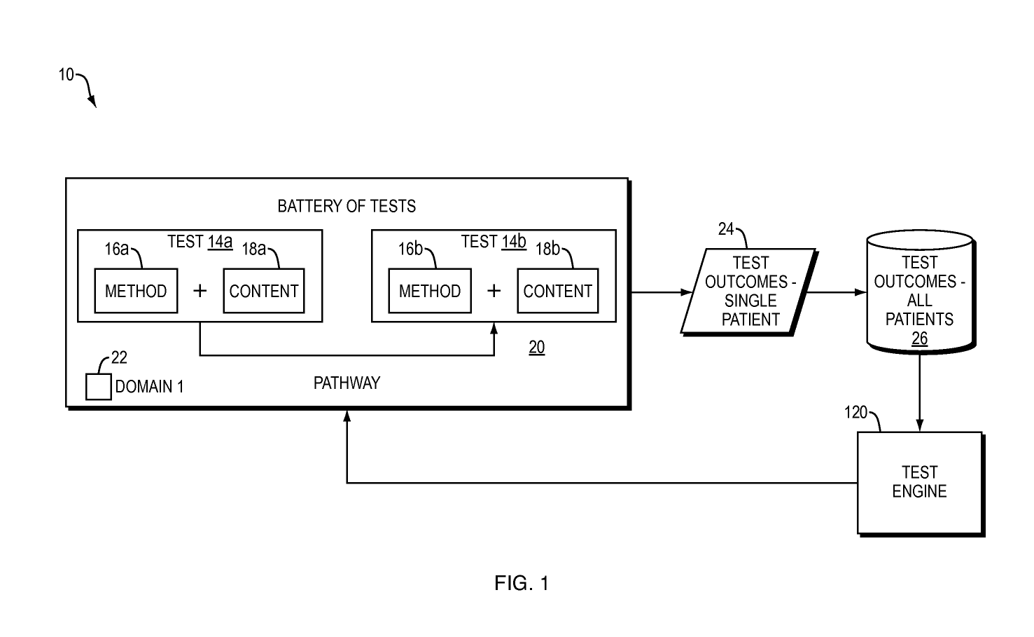

Referring to FIG. 1, a dataflow diagram is shown of a system 10 for dynamically adapting tests according to one embodiment of the present invention. For purposes of example, data 14a and 14b representing two tests are shown in FIG. 1. Although FIG. 1 shows the data 14a and 14b that represent a first test and a second test, respectively, the description herein may refer to elements 14a and 14b as “tests,” rather than as “data representing tests,” for ease of explanation. Furthermore, although only two tests 14a-b are shown in FIG. 1 for ease of illustration, the system 10 may include any number of tests. The test data 12a-b may be stored in any suitable form on one or more non-transitory computer-readable media.

The system 10 includes a battery of tests 12, which in the example of FIG. 1 includes tests 14a-b. In general, the term “battery of tests” refers herein to a group of tests that are targeted at a particular type of patient in a diagnostic process. For example, the battery of tests 12 may be targeted at patients who are scheduled for heart surgery. Although the particular battery of tests 12 shown in FIG. 1 contains two tests 14a-b, more generally a battery of tests may include any number of tests (including one). As shown in the example of FIG. 1, the first test 14a includes its own method 16a and content, and the first test 14b includes its own method 16b and content 18b. The methods 16a and 16b may be the same as or differ from each other. Similarly, the contents 18a and 18b may be the same as or differ from each other. Although not shown in FIG. 1, other batteries of tests may include tests that differ from the tests 14a-b of battery 12 in their methods and/or content.

The term “pathway” as used herein refers to a particular sequencing of tests within a particular battery of tests. For example, battery of tests 12 includes pathway 20, which specifies that test 14a is to be performed first in the battery of tests 12, and that test 14b is to be performed after the completion of test 14a. Pathways may specify other types of sequencing in addition to or instead of the simple sequence of pathway 20. For example, a pathway may specify that tests (or sequences of tests) are to be repeated some number of times (e.g., some fixed number of times or until some predetermined condition is satisfied). As another example, a pathway may include conditional sequencing, such that if a particular specified condition is satisfied, then a first sequence of tests is performed, while if the particular specified condition is not satisfied, then a second sequence of tests is performed.

The battery of tests 12 also includes a domain 22, which represents the domain that is tested by the battery of tests 12. Although the domain may remain fixed throughout all of the tests 14a-b within the battery of tests 12, alternatively the domain 22 may vary among tests 14a-b within the battery of tests 12. For example, the pathway 20 may specify how the domain 22 is to change from one test to another within the battery of tests 12.

The term “cohort” refers herein to a group of patients who have some set of characteristics that have been determined to be sufficiently similar to each other according to some set of criteria. Such characteristics may, for example, include any one or more of test results, clinical outcomes, test outcomes, demographic data, and clinical data. The term “risk cohort” refers herein to a group of patients who have similar risk profiles. In general, a patient's risk profile lists the risks associated with each of a plurality of clinical outcomes for that patient.

In general, embodiments of the present invention may be used to adapt batteries of tests and any components thereof (such as tests, methods, contents, pathways, and domains). Therefore, any reference herein to adapting a battery of tests should be understood to refer to adapting a battery of tests or any component thereof. The term “adapt,” as used herein includes both selecting an existing battery, test, method, content, pathway, or domain, and modifying an existing battery, test, method, content, pathway, or domain to produce a modified version thereof.

The system 10 includes a test engine 120, which may perform such adaptation based on any of a variety of data. For example, the test engine 120 may receive as input any one or more of the EOC data, external data, EOC model data, prediction/recommendation data, EOC database data, as described in the above-referenced U.S. Prov. Pat. App. Ser. No. 61/714,106. The engine 120 may adapt the battery of tests 12 based on any such data, individually or in any combination.

The engine 120 may perform adaptation on the battery of tests 12 before, during, or after performance of any of the tests 14a-b within the battery of tests 12. For example, the engine 120 may select the battery of tests 12 from among a plurality of batteries of tests (not shown) based on any of the data described herein, and then perform the selected battery of tests 12. As another example, the engine 120 may perform the first test 14a within the battery of tests 12 and then select a test (not shown) other than the test 14b originally contained within the battery of tests 12 based on any of the data described herein, and then perform the selected test, rather than the originally-specified test 14b. As another example, the engine 120 may begin to perform the test 14a and then, based on any of the data described herein (such as partial outcomes of the test 14a), the engine 120 may modify the test 14a, such as by modifying the method 16a and/or content 18a of the test. The engine 120 may then continue performing the test 14a in its modified form. As another example, the engine 120 may perform the test 14a and then, based on any of the data described herein, modify the next test 14b, and then perform the test 14b in its modified form.

As a concrete example of this general method, considering the following. A first patient arrives at a hospital for a scheduled surgery. Hospital staff perform a pre-surgical test on the first patient (which is an example of the first test 14a of the battery of tests 12) and a post-surgical test on the first patient (which is an example of the second test 14b of the battery of tests 12). Assume that the purpose (i.e., domain 22) of the battery of tests 12 is to determine whether the first patient's physical and neurocognitive rehabilitation is progressing as expected.

The engine 120 may evaluate the results 14 of the battery of tests 12 in any of a variety of ways. For example, the engine 120 may compare the results of the first test 14a to the results of the second test 14b to determine whether the first patient's physical and neurocognitive functions have changed after the surgery relative to their baseline level before the surgery. The engine 120 may even have modified the second test 14b before it was performed based on the results of the first test 14a.

To evaluate the significance, if any, of any difference between the results of the first test 14a and the results of the second test 14b, the engine 120 may, for example, compare the results of the battery of tests 12 to results from the same battery of tests 12 when applied to EOC cohorts, e.g., patients who have undergone the same or similar EOCs as the first patient. Such EOC cohorts may further be narrowed based on, for example, demographic data, to include only EOCs for patients who are demographically similar to the first patient. As another example, the EOC cohorts may further be narrowed based on contextual data to include only EOCs for patients who experienced the EOC in contexts that were similar to the context in which the first patient experienced the EOC (e.g., similar hospitals, dates, or times of year). The engine 120 may then identify any differences between the first patient's test outcomes 24 and the test outcomes 26 of patients in the EOC cohort and determine whether any such differences are significant.

The example above is merely one example of a way in which the engine 120 may take into account the first patient's EOC when determining whether and how to modify the battery of tests 12 that is performed on the first patient. In particular, an EOC cohort for the first patient may be developed by identifying associations (e.g., correlations) between the first patient's EOC model and the EOC models of other patients and thereby identifying EOC models that are sufficiently similar to the first patient's EOC model as the first patient's EOC cohort. By then identifying associations (e.g., correlations) between the outcomes 24 of the first patient's battery of tests 12 and outcomes 26 of batteries of tests performed on patients in the EOC cohort as part of their similar EOCs, the engine 120 indirectly takes into account the first patient's EOC model when determining whether and how to modify the first patient's battery of tests 12.

Having described certain features of embodiments of the present invention in general, certain systems and methods that may be used to implement embodiments of the present invention will now be described.

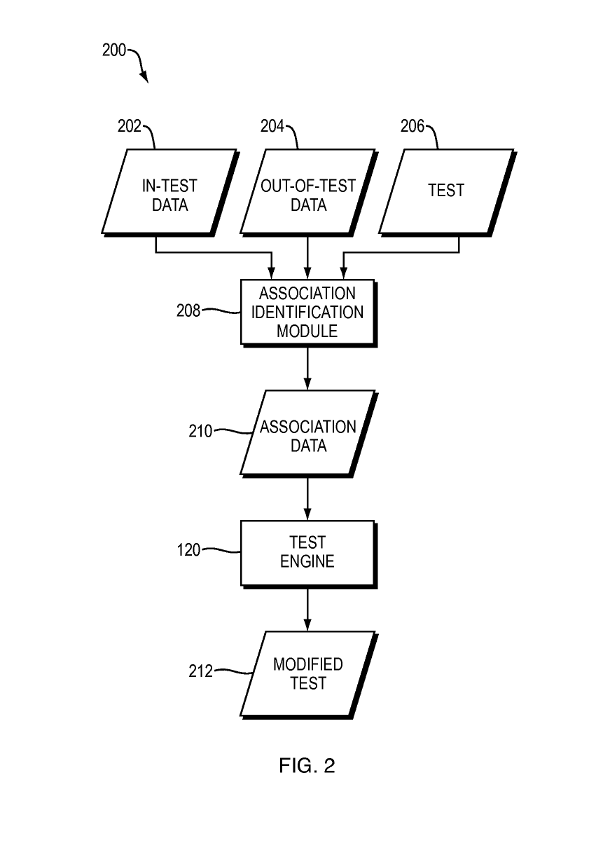

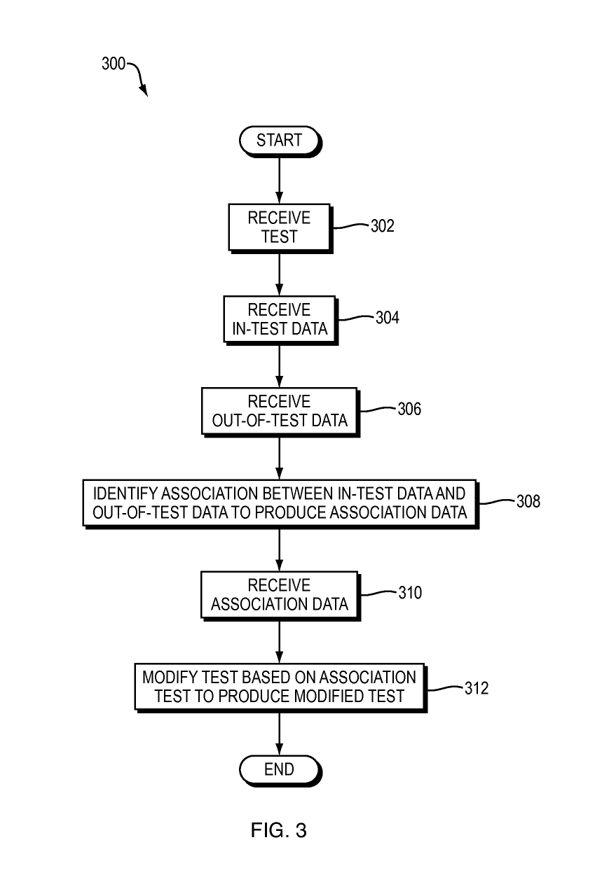

Referring to FIG. 2, a dataflow diagram is shown of a system 200 for adapting medical tests according to one embodiment of the present invention. Referring to FIG. 3, a flowchart is shown of a method 300 performed by the system 200 of FIG. 2 according to one embodiment of the present invention.

The system 200 includes a test 206, such as the test 14a or 14b of FIG. 1. Although the system 200 and method 300 of FIGS. 2 and 3 are described herein as operating on the test 206, more generally the techniques of FIGS. 2 and 3 may be applied to multiple tests, such as a battery of tests (e.g., the battery of tests 20 of FIG. 1).

The system 200 also includes an association identification module 208. The association identification module 208 receives the following inputs: (1) the test 206 (FIG. 3, operation 302); (2) in-test data 202 (FIG. 3, operation 304); and (3) out-of-test data 204 (FIG. 3, operation 306). The association identification module 208 identifies an association between the in-test data 202 and the out-of-test data 204 (FIG. 3, operation 308), thereby producing association data 210, which represents the association identified by the association identification module 208.

The system 200 also includes the test engine 120 of FIG. 1. The test engine receives the association data 210 as input (FIG. 3, operation 310) and modifies the test 206 based on the association data 210, thereby producing a modified test 212 which differs from the original test 206 (FIG. 3, operation 312).

The in-test data 202 may include any of a variety of data obtained by the association identification module 208 during performance of the test 206. For example, the in-test data 202 may include sensor data received via a sensor from the patient on whom the test 206 is being performed. Examples of this include receiving accelerometer data from the patient via an accelerometer during performance of the test 206, receiving image data from the patient via an image sensor during performance of the test 206, and receiving audio data from the patient via an audio sensor during performance of the test.

The out-of-test data 204 may include any of a variety of data obtained by the association identification module 208 from outside performance of the test 206. For example, the out-of-test data 204 may include any one or more of the following: contextual data, medical history data, clinical data, symptom/disease/syndrome data, patient profile data, organization data, template of care data, workflow data, epoch of care cohort data, and epoch of care data, as those terms are used in the above-referenced U.S. Prov. Pat. App. Ser. No. 61/714,106. For example, the out-of-test data 204 may include some or all of an epoch of care model of the patient on whom the test 206 is performed. The test 206 may be performed within the epoch of care represented by the epoch of care model of the patient on whom the test 206 is performed.

The association identification module 208 may identify the association between the in-test data 202 and the out-of-test data 204 in any of a variety of ways. For example, the association identification module 208 may identify the association between the in-test data 202 and the out-of-test data 204 by correlating the in-test data 202 with the out-of-test data 204, in which case the association data 210 may include correlation data representing the results of the correlation. The test engine 120 may then adapt the test 206 to produce the modified test 212 based on the correlation data.

The association identification module 208 may obtain the in-test data 202 while the test 206 is being performed on the patient. For example, the association identification module 208 may obtain the in-test data 202 in real-time, e.g., from a sensor. The test engine 120 may adapt the test 206 to produce the modified test 212 while the test 206 is being performed on the patient, e.g., in real-time.

As mentioned above, the test 206 may be one test in a battery of tests. In such a case, the test engine 120 may modify the battery of tests by, for example, adding a test to the battery of tests, removing a test (e.g., the test 206) from the battery of tests, or modifying a pathway in the battery of tests.

The test engine 120 may, for example, modify the test 206 to produce the modified test 212 by modifying a method of the test 206, thereby producing a modifying method within the modified test 212. Examples of modifying a method within the test 206 include modifying technology that is used to provide test input to the patient during the test, modifying media through which content of the test is provided to the patient, modifying a presentation quality of the test, and modifying technology that contributes to ambient features of an environment of the test.

As another example, the test engine 120 may modify the test 206 to produce the modified test 212 by modifying content of the test 206, thereby producing modified content within the modified test 212. As yet another example, the test engine 120 may modify the test 206 to produce the modified test 212 by modifying content of the test 206, thereby producing modified content within the modified test 212.

Having described certain features of embodiments of the present invention in general, features of particular embodiments of the present invention will now be described in more detail.

As described above, the test engine 120 may adapt the battery of tests 12 based on any of the data disclosed herein. As one particular example, the test engine 120 may adapt the battery of tests based on the sensor data 108f, which are data obtained from the first patient via sensors. For example, the first patient may provide test input to the tests 14a-b in the battery of tests 12 via sensors, thereby generating sensor data 108f. For example, if the test 14a is a handwriting test and the first patient writes a spoken sentence on the touchscreen of a tablet computer using a stylus, then the first patient's handwriting input is an example of the sensor data 108f. As another example, if the test 14a is a test of the first patient's balance which uses the accelerometer of a tablet computer to measure how level the first patient holds the tablet computer over a period of time, then the output of the accelerometer is an example of sensor data 108f.

As these examples illustrate, multiple units of sensor data 108f may be generated in response to a single test. For example, if the test 14a contains ten questions, then the answers provided by the first patient to the ten questions may constitute ten units of sensor data 108f, all of which are answers (inputs) to the same test 14a. As another example, the accelerometer data described above may contain a large number of bytes of data arranged in a stream or other data structure, all of which may constitute sensor data 108f generated in response to the same test 14a.

The test engine 120 may identify associations (e.g., correlations) between sensor data 108f and outcome data 24, and adapt the battery of tests 12 based on the identified associations. The sensor data 108f may include sensor data 108f generated from the performance of any number of batteries of tests, each of which may be performed on any number of people any number of times. For example, the sensor data 108f may include sensor data generated from:
a single run of the battery of tests 12 on a single patient;
multiple runs of the same battery of tests 12 on a single patient (e.g., during different visits on different dates);
runs of the same battery of tests 12 on multiple patients (e.g., including multiple runs of the same battery of tests 12 on each of the multiple patients);
runs of different batteries of tests on a single patient (e.g., including multiple runs of each of multiple batteries of tests on the single patient); and
runs of different batteries of tests on multiple patients (e.g., including multiple runs of each of multiple tests on each of the multiple patients).

The outcome data 24 may include outcome data from any of the kinds of sensor data described above.

The test engine 120 may identify associations (e.g., correlations) between any of the sensor data 108f and any of the outcome data 24 described above. For example, the test engine 120 may correlate sensor data 108f from runs of the same battery of tests 12 on multiple patients with the outcome data 14 generated by that sensor data 108f. The test engine 120 may adapt the battery of tests 12 based on the results of such correlation.

The test engine 120 may identify associations (e.g., correlations) between sensor data 108f and both outcome data 14 and any of the other data disclosed herein. For example, the test engine 120 may identify associations between sensor data 108f and both outcome data 24 and any one or more of contextual data, profile data, medical history data, organization data, symptom/disease/syndrome data, demographic data, coordination data, patient database data, structured EOC data, unstructured contextual data, EOC data, external data, EOC model data, prediction/recommendation data, EOC database data, EOC cohort data, template of care data, and workflow data, as those terms are used in the above-referenced U.S. Prov. Pat. App. Ser. No. 61/714,106. The test engine 120 may then adapt the battery of tests 12 in any of the ways disclosed herein.

Embodiments of the present invention have a variety of advantages, such as the following.

One benefit of embodiments of the present invention is that they may be used to dynamically adapt batteries of tests while they are being performed on patients. In the traditional practice of medicine, a battery of tests is selected to perform on a patient based on a limited set of factors such as the patient's symptoms and known illnesses. Once the battery of tests is selected, however, the battery of tests is performed on the patient without modification. In contrast, embodiments of the present invention may dynamically adapt the battery of tests that is performed on a patient while the battery of tests is being performed on the patient, e.g., in real-time, in response to sensor data obtained from the patient via the battery of tests itself, possibly in combination with other data (such as data obtained outside the battery of tests). For example, tests may be added to and/or removed from the battery of tests while the battery of tests is being performed. As another example, the methods and/or contents of tests within the battery of tests may be modified while the battery of tests is being performed. As another example, the pathway of the battery of tests may be modified while the battery of tests is being performed. As yet another example, the domain of the battery of tests may be modified while the battery of tests is being performed. In all of these cases, the ability to adapt the battery of tests dynamically while it is being performed, in response to sensor data obtained within the battery of tests and possibly also in response to other data, enables embodiments of the present invention to provide tests that are more likely to obtain data that may be used to accurately diagnose and assess the patient than traditional methods which rely on static tests that are based on more generic information.

A related advantage of embodiments of the present invention is the ability to adapt a battery of tests based on sensor data obtained from real-time sensors such as accelerometers, handwriting sensors (e.g., tablet computers with a touchscreen and stylus), and speech sensors (e.g., tablet computers with a microphone and speech recognition software). Embodiments of the present invention may use such sensor data both to obtain sensor data in real-time and to adapt the battery of tests in real-time. Previous systems either lacked the ability to obtain such real-time sensor data due to the lack of appropriate sensors or lacked the ability to analyze such data to dynamically adapt the battery of tests in real-time based on such data. Embodiments of the present invention include the ability both to obtain sensor data in real-time and to dynamically adapt the battery of tests based on such sensor data in real-time. As a result, embodiments of the present invention enable batteries of tests to be adapted, e.g., in real-time, based on sensor data obtained in real-time.

Another benefit of embodiments of the present invention is that they enable a battery of tests to be adapted based on a combination of data obtained within the test (e.g., sensor data) and data not obtained within the test, such as any one or more of epoch of care data, clinical data, and contextual data. Although sensor data obtained within the battery of tests may be used for adapting the battery of tests, relying on such data alone may result in adaptations that do not take into account factors that are not measured by the battery of tests itself, such as the age of the patient, future events in the patient's epoch of care (e.g., a scheduled surgery), and tests that have proven useful to perform on patients who have undergone similar epochs of care, as evidenced by a strong correlation between performance of those other tests and positive outcomes for the patients with similar epochs of care. By taking into account a combination of in-test and out-of-test data, embodiments of the present invention may be used to produce test adaptations that respond dynamically to the patient's test input in real-time, while also taking into account the broader context in which the battery of tests is being performed. In particular, embodiments of the present invention may be used to adapt the patient's battery of tests in a way that is tailored to the patient's particular epoch of care.

As described above, embodiments of the present invention may determine whether and how to adapt the battery of tests by identifying associations (e.g., correlations) between the sensor data obtained within the test and outcomes of the test, possibly in combination with other data. Because such associations do not assume any particular relationship among data, embodiments of the present invention may discover associations between sensor data, outcome data, and other data that previously were unknown. For example, embodiments of the present invention may discover that performance of a particular test is strongly correlated with decreased risk of chest pain after heart surgery among patients who are over 60 years old. As a result of such a discovery, embodiments of the present invention may recommend that the particular test be performed on patients who are over 60 years old and who are scheduled for heart surgery.

Similarly, embodiments of the present invention may discover not only associations that were previously undiscovered, but also associations that were previously undiscoverable due to the unavailability and/or lack of use of particular sensors in tests, such as accelerometers. For example, if an embodiment of the present invention performs a test in which a patient's balance is tested using an accelerometer which generates a stream of real-time accelerometer output, then the embodiment of the present invention may discover, by correlating the accelerometer output with the outcome of ear surgery that patients who exhibit poor balance before ear surgery are more likely to suffer complications after the ear surgery than patients who exhibit good balance before ear surgery. As this example illustrates, embodiments of the present invention may be used to discover associations between previously unused types of sensor data and patient outcomes.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

What is claimed is:

1. A method performed by at least one computer processor executing computer program instructions stored on at least one non-transitory computer-readable medium, the method comprising:
(A) receiving first sensor data from a patient via a sensor substantially in real-time while a test is being performed on the patient for at least one of diagnosing a disease of the patient and assessing an outcome of the patient,
wherein the first sensor data comprises at least one of accelerometer data received via the sensor, velocity data received via the sensor, and pressure data received via the sensor,
wherein the test is represented by test data stored in the at least one non-transitory computer-readable medium;
wherein the test data comprises method data representing a method of the test, content data representing content of the test, and domain data representing a domain of the test;
(B) receiving data obtained outside the test;
(C) identifying an association between the sensor data and the data obtained outside the test;
(D) modifying at least one of the method data, the content data, and the domain data, substantially in real-time and while the test is being performed on the patient, based on the identified association, to produce modified test data representing a modified form of the test and stored in the at least one non-transitory computer-readable medium; and
(E) receiving second sensor data from the patient via the sensor substantially in real-time while the modified test is being performed on the patient.

2. The method of claim 1, wherein (C) comprises correlating the sensor data with the data obtained outside the test to produce correlation data, and wherein (D) comprises modifying the test based on the correlation data.

3. The method of claim 1, wherein the data obtained outside the test comprises epoch of care data representing an epoch of care within which the test is performed.

4. The method of claim 1, wherein the data obtained outside the test comprises contextual data.

5. The method of claim 1, wherein the data obtained outside the test comprises clinical data.

6. The method of claim 1, wherein the data obtained outside the test comprises epoch of care cohort data.

7. The method of claim 1, wherein the data obtained outside the test comprises profile data of the patient.

8. (canceled)

9. (canceled)

10. The method of claim 1, wherein the test comprises a battery of tests, and wherein (D) comprises modifying the test data to represent the addition of another test to the battery of tests.

11. The method of claim 1, wherein the test comprises a battery of tests, and wherein (D) comprises modifying the test data to represent the removal of a test from the battery of tests.

12. The method of claim 1, wherein the test comprises a battery of tests, and wherein (D) comprises modifying the test data to represent a modification to a pathway of the battery of tests.

13. The method of claim 1, wherein (D) comprises modifying the test data to represent a modification to the method of the test.

14. The method of claim 13, wherein the test data comprises data representing technology that is used to provide test input to the patient during the test, and wherein (D) comprises modifying the test data to represent a modification to the technology that is used to provide test input to the patient during the test.

15. The method of claim 1, wherein (D) comprises modifying the test data to represent a modification to the content of the test.

16. The method of claim 1, wherein (D) comprises modifying the test data to represent a modification to the domain of the test.

17. (canceled)

18. The method of claim 1, wherein (A) further comprises receiving image data from the patient via an image sensor during the test substantially in real-time.

19. The method of claim 1, wherein (A) further comprises receiving audio data from the patient via an audio sensor during the test substantially in real-time.

20. A system comprising at least one non-transitory computer-readable medium containing computer program instructions, wherein the computer program instructions are executable by at least one computer processor to perform a method, the method comprising:
(A) receiving first sensor data from a patient via a sensor substantially in real-time while a test is being performed on the patient for at least one of diagnosing a disease of the patient and assessing an outcome of the patient,
wherein the first sensor data comprises at least one of accelerometer data received via the sensor, velocity data received via the sensor, and pressure data received via the sensor,
wherein the test is represented by test data stored in the at least one non-transitory computer-readable medium;
wherein the test data comprises method data representing a method of the test, content data representing content of the test, and domain data representing a domain of the test;
(B) receiving data obtained outside the test;
(C) identifying an association between the sensor data and the data obtained outside the test;
(D) modifying at least one of the method data, the content data, and the domain data, substantially in real-time and while the test is being performed on the patient, based on the identified association, to produce modified test data representing a modified form of the test and stored in the at least one non-transitory computer-readable medium; and
(E) receiving second sensor data from the patient via the sensor substantially in real-time while the modified test is being performed on the patient.

21. The method of claim 1, wherein the method of the test comprises a technology for providing the content of the test to the patient, and wherein (D) comprises modifying data representing the technology.

22. The method of claim 1, wherein the method of the test comprises a medium for providing the content of the test to the patient, and wherein (D) comprises modifying data representing the medium.

23. The method of claim 1, wherein the content of the test comprises a question, and wherein (D) comprises modifying data representing the question.

24. The method of claim 1, wherein the content of the test comprises an instruction, and wherein (D) comprises modifying data representing the instruction.
